(19) United States
(12) Patent Application Publication
(10) Pub. No.: US 2024/0190896 A1
(43) Pub. Date: Jun. 13, 2024

(54) SMTP-7 DERIVATIVE AND USE THEREOF

(71) Applicants: Shanghai Senhui Medicine Co., Ltd., Shanghai (CN); Shanghai Shengdi Pharmaceutical Co., Ltd., Shanghai (CN); Jiangsu Hengrui Pharmaceuticals Co., Ltd., Lianyungang, Jiangsu (CN)

(72) Inventors: Lingjian Zhu, Shanghai (CN); Xiuzhao Yu, Shanghai (CN); Xiaowu Liu, Shanghai (CN); Peipei Ben, Lianyungang, Jiangsu (CN); Lei Chen, Lianyungang, Jiangsu (CN); Xianglin Kong, Lianyungang, Jiangsu (CN); Jian Huang, Shanghai (CN); Piaoyang Sun, Lianyungang, Jiangsu (CN)

(21) Appl. No.: 18/546,071

(22) PCT Filed: Feb. 10, 2022
(86) PCT No.: PCT/CN2022/075740

§ 371 Date: Aug. 10, 2023

(30) Foreign Application Priority Data
Feb. 10, 2021 (CN) 202110184080.0
Dec. 10, 2021 (CN) 202111503546.5
Dec. 31, 2021 (CN) 202111664079.4

(51) Int. Cl. C07D 519/00; A61K 31/407; A61P 9/10; C07B 59/00

(52) U.S. Cl. CPC C07D 519/00; A61K 31/407; A61P 9/10; C07B 59/002; C07B 2200/05

(57) ABSTRACT

The present disclosure relates to an SMTP-7 derivative and the use thereof. In particular, provided is a compound as represented by formula I or a pharmaceutically acceptable salt thereof, wherein each group is as defined in the description.

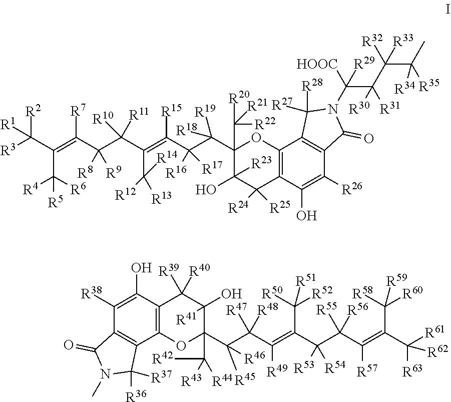

### TECHNICAL FIELD

The disclosure belongs to the field of pharmaceutics, and relates to an SMTP-7 derivative and use thereof.

### BACKGROUND

SMTP-7 (TMS-007, Stachybotrys microspore triprenyl phenol-7) was extracted from a mold (Stachybotrys microspora) on a special kind of fallen leaves on Iriomote Island of Okinawa prefecture in 2000. It is a small-molecule plasminogen activator and is similar to vitamin E in structure. It breaks down blood clots through a novel mechanism of action and is also believed to be capable of inhibiting local inflammation at the site of a thrombus. In addition, SMTP-7 has anti-tumor angiogenesis activity, anti-oxidation activity and pro-tissue regeneration activity (WEIMIN H, SHIGEKI O, et al. J. Antibiot., 2000, 53(3): 241-247).

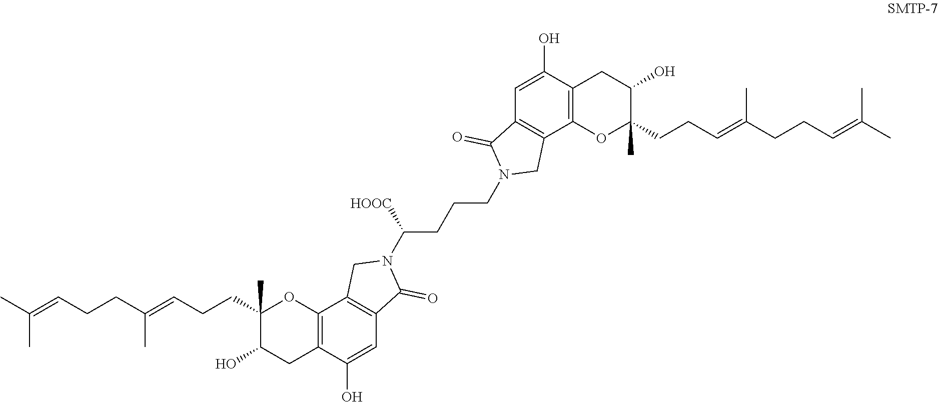 [SMILES: CC(C)=CCC/C(C)=C/CC[C@]1(C)Oc2c(c(O)cc3c2CN(CCC[C@@H](C(=O)O)N2Cc4c(cc(O)c5c4O[C@@](C)(CC/C=C(\C)CCC=C(C)C)[C@@H](O)C5)C2=O)C3=O)C[C@@H]1O]

Plasminogen is a plasmin precursor, which can be activated to produce plasmin. This is a protease, which can hydrolyze many proteins, including thrombospondin. Upon binding to plasminogen, SMTP-7 changes its molecular conformation so that it is more easily activated by the plasminogen activator. Therefore, SMTP-7 per se does not have the function of activating plasminogen but makes the activation process easier. The unique combined action of SMTP-7 makes it possible for SMTP-7 to become a best-in-class thrombolytic drug for treating acute ischemic stroke (AIS). Compared to the existing standard thrombolytic drugs, SMTP-7 has the potential to prolong the therapeutic window (while many antihypertensive, lipid-lowering and anticoagulant drugs can prevent strokes, the only therapeutic drug is the recombinant tissue plasminogen activator (rt-PA, alteplase) for ischemic strokes, whose major ingredient is a glycoprotein containing 526 amino acids). The SMTP molecule induces a change in the conformation of plasminogen, resulting in accelerated binding of plasminogen to fibrin and finally activation leading to plasmin. In addition, SMTP induces plasmin to self-cleave to provide angiogenic human angiostatin-like fragments. This activity is believed to be the mechanism of the anti-angiogenesis and anti-tumor effects of SMTP molecules. In addition, SMTP-induced increases in activated plasminogen may control local extracellular proteolysis, thereby leading to tissue remodeling, wound healing and tissue regeneration.

### SUMMARY

The disclosure provides a compound of formula I or a pharmaceutically acceptable salt thereof,

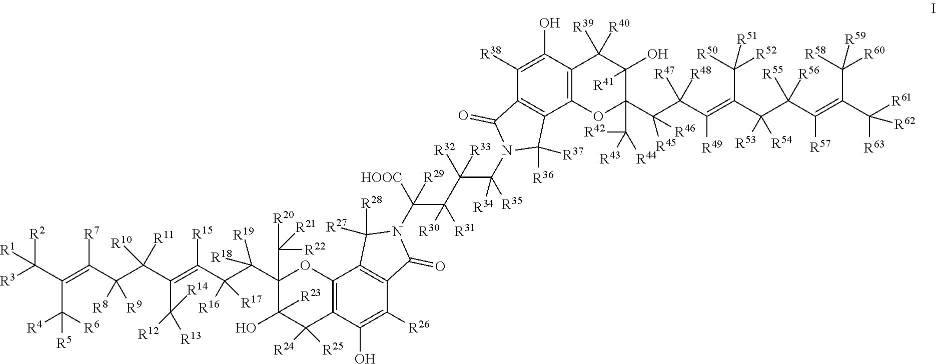

wherein R¹, R², R³, R⁴, R⁵, R⁶, R⁷, R⁸, R⁹, R¹⁰, R¹¹, R¹², R13 , R14 , R¹⁵, R¹⁶, R¹⁷, R¹⁸, R¹⁹,R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶² and R⁶³ are each independently hydrogen or deuterium, and at least one of R¹, R², R³, R⁴, R⁵, R⁶, R⁷, R⁸, R⁹, R¹⁰⁻, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶²and R⁶³ is deuterium.

In some embodiments, in the compound of formula I or the pharmaceutically acceptable salt thereof, R²⁷ is selected from deuterium; R²⁸ is selected from deuterium; R³⁶ is selected from deuterium; R³⁷ is selected from deuterium.

In some embodiments, in the compound of formula I or the pharmaceutically acceptable salt thereof, R²⁶ is selected from deuterium; R³⁸ is selected from deuterium.

In some embodiments, in the compound of formula I or the pharmaceutically acceptable salt thereof, R²⁴ is selected from deuterium; R²⁵ is selected from deuterium; R³⁹ is selected from deuterium; R⁴⁰ is selected from deuterium.

In some embodiments, in the compound of formula I or the pharmaceutically acceptable salt thereof, R²⁹ is selected from deuterium.

In some embodiments, in the compound of formula I or the pharmaceutically acceptable salt thereof, R³⁰ is selected from deuterium; R³¹ is selected from deuterium.

In some embodiments, in the compound of formula I or the pharmaceutically acceptable salt thereof, R³⁴ is selected from deuterium; R³⁵ is selected from deuterium.

In some embodiments, in the compound of formula I or the pharmaceutically acceptable salt thereof, R¹⁶ is selected from deuterium; R¹⁷ is selected from deuterium; R⁴⁷ is selected from deuterium; R⁴⁸ is selected from deuterium.

In some embodiments, in the compound of formula I or the pharmaceutically acceptable salt thereof, R¹² is selected from deuterium; R¹³ is selected from deuterium; R¹⁴ is selected from deuterium.

In some embodiments, in the compound of formula I or the pharmaceutically acceptable salt thereof, R⁵⁰ is selected from deuterium; R⁵¹ is selected from deuterium; R⁵² is selected from deuterium.

In some embodiments, in the compound of formula I or the pharmaceutically acceptable salt thereof, R²³ is selected from deuterium; R⁴¹ is selected from deuterium.

In some embodiments, in the compound of formula I or the pharmaceutically acceptable salt thereof, R³² is selected from deuterium; R³³ is selected from deuterium.

In some embodiments, in the compound of formula I or the pharmaceutically acceptable is salt thereof, R³² is selected from deuterium; R³¹ is selected from deuterium; R³² is selected from deuterium; R³³ is selected from deuterium; R³⁴ is selected from deuterium; R³⁵ is selected from deuterium.

In some embodiments, in the compound of formula I or the pharmaceutically acceptable salt thereof, R²⁹ is selected from deuterium; R³⁰ is selected from deuterium; R³¹ is selected from deuterium; R³² is selected from deuterium; R³³ is selected from deuterium; R³⁴ is selected from deuterium; R³⁵ is selected from deuterium.

In some embodiments, in the compound of formula I or the pharmaceutically acceptable salt thereof, R¹ is selected from deuterium; R² is selected from deuterium; R³ is selected from deuterium.

In some embodiments, in the compound of formula I or the pharmaceutically acceptable salt thereof, R⁴ is selected from deuterium; R⁵ is selected from deuterium; R⁶ is selected from deuterium.

In some embodiments, in the compound of formula I or the pharmaceutically acceptable salt thereof, R⁵⁸ is selected from deuterium; R⁵⁹ is selected from deuterium; R⁶⁰ is selected from deuterium.

In some embodiments, in the compound of formula I or the pharmaceutically acceptable salt thereof, R⁶¹ is selected from deuterium; R⁶² is selected from deuterium; R⁶³ is selected from deuterium.

In some embodiments, in the compound of formula I or the pharmaceutically acceptable salt thereof, R¹ is selected from deuterium; R² is selected from deuterium; R³ is selected from deuterium; R⁴ is selected from deuterium; R⁵ is selected from deuterium; R⁶ is selected from deuterium; R⁵⁸ is selected from deuterium; R⁵⁹ is selected from deuterium; R⁶⁰ is selected from deuterium; R⁶¹ is selected from deuterium; R⁶² is selected from deuterium; R⁶³ is selected from deuterium.

In some embodiments, in the compound of formula I or the pharmaceutically acceptable salt thereof, R⁷ is selected from deuterium; R⁵⁷ is selected from deuterium.

In another aspect, the compound of formula I or the pharmaceutically acceptable salt thereof provided in some embodiments is

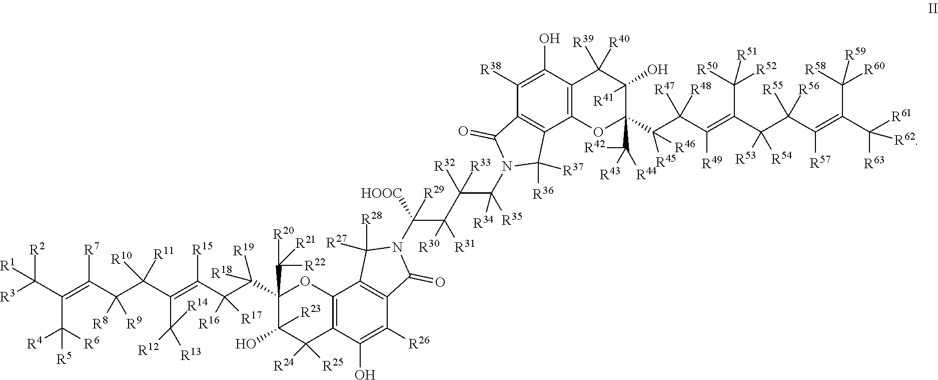

Typical compounds of formula I include, but are not limited to:

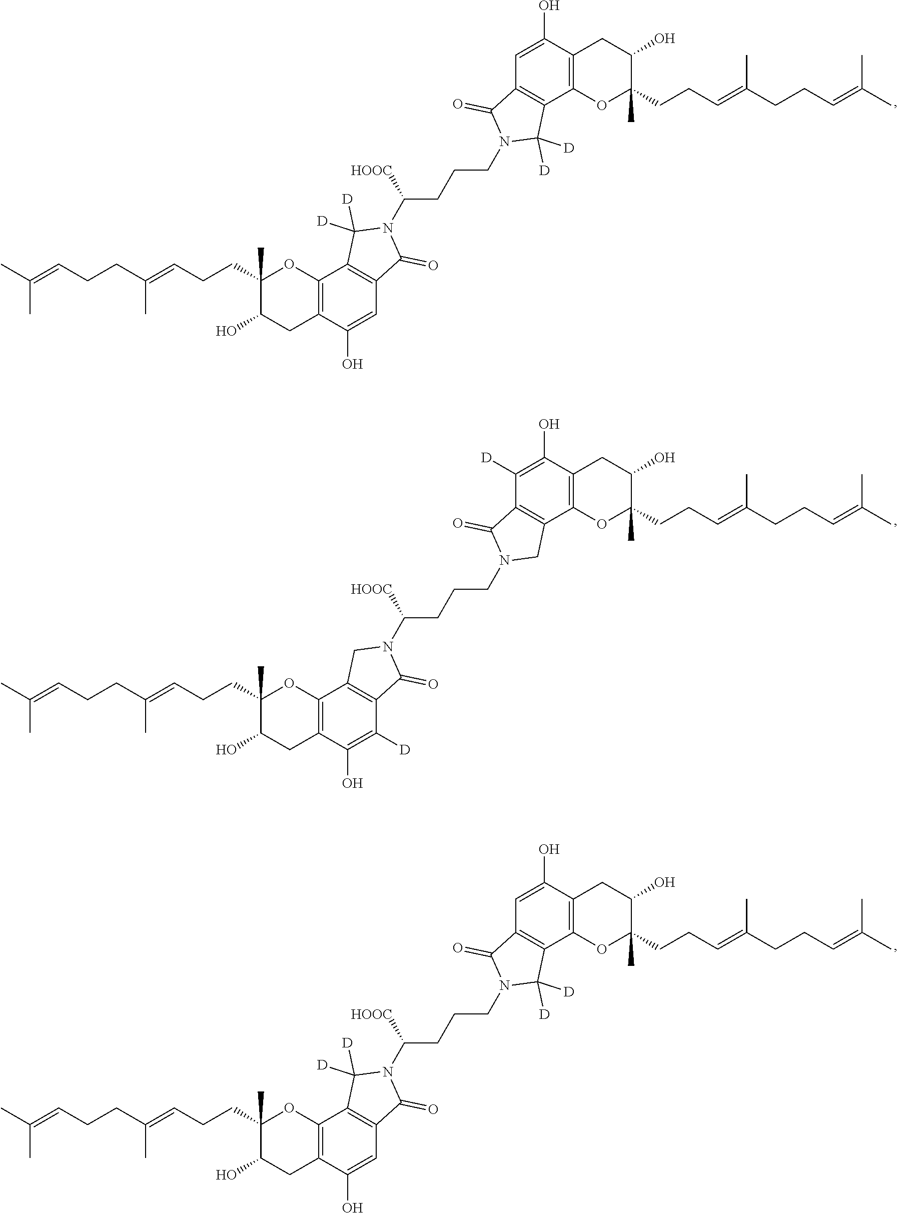

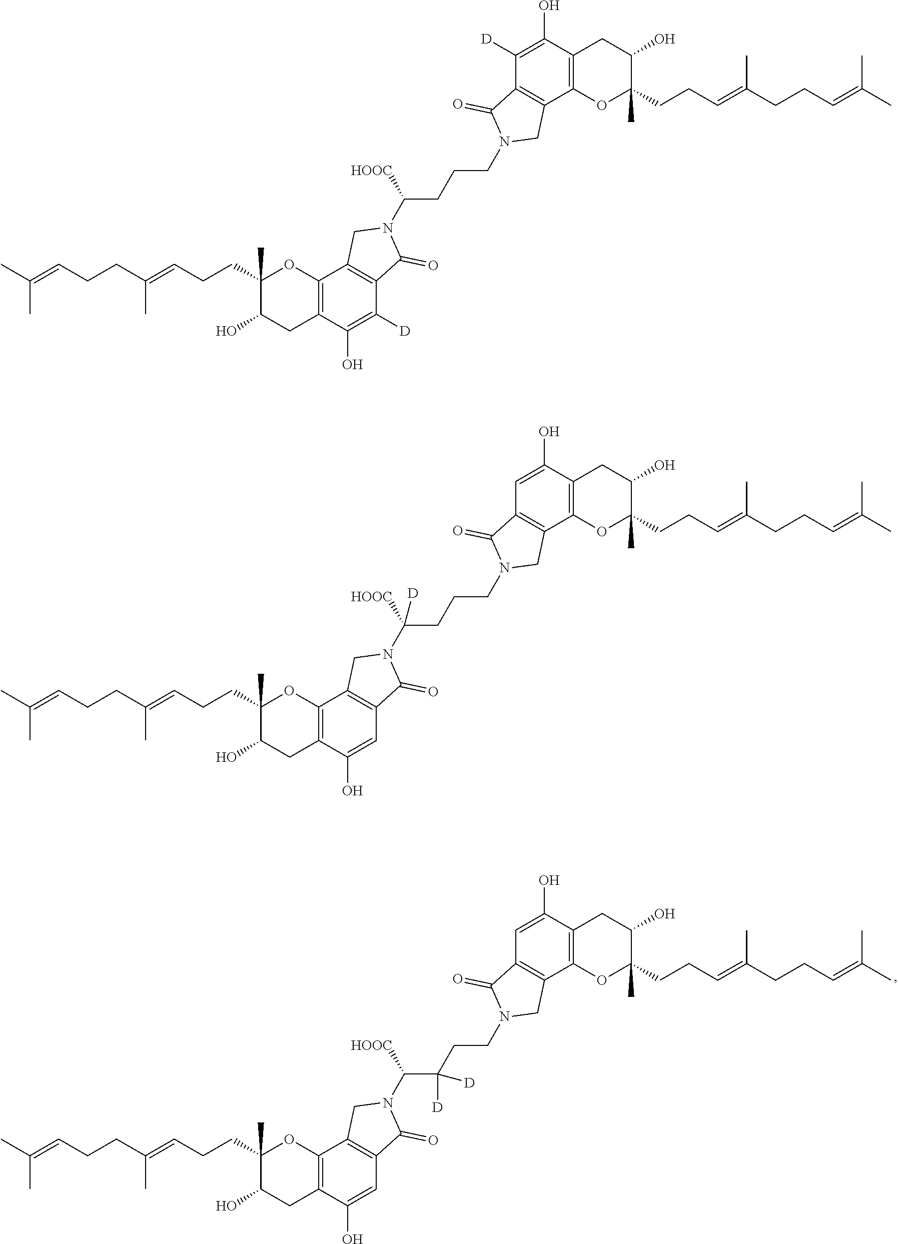

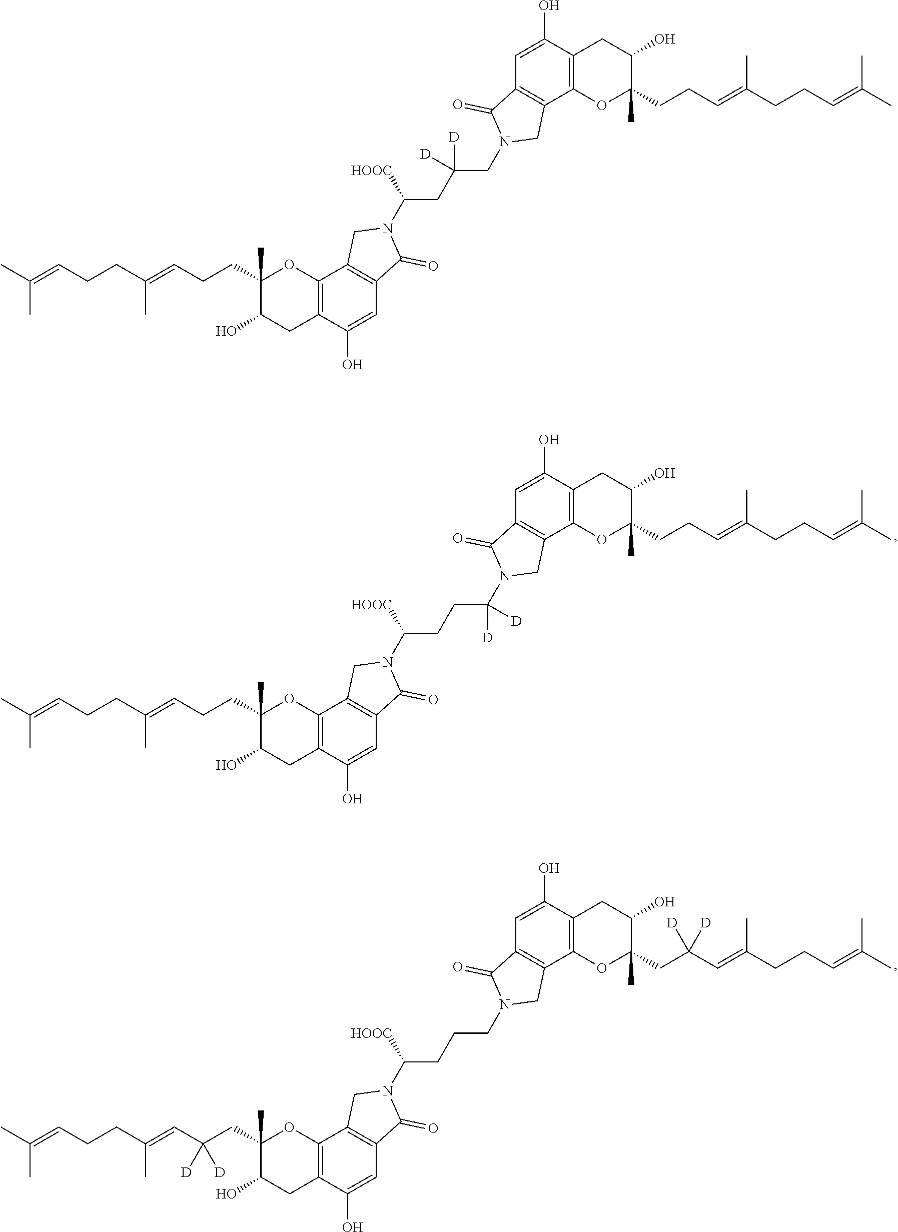

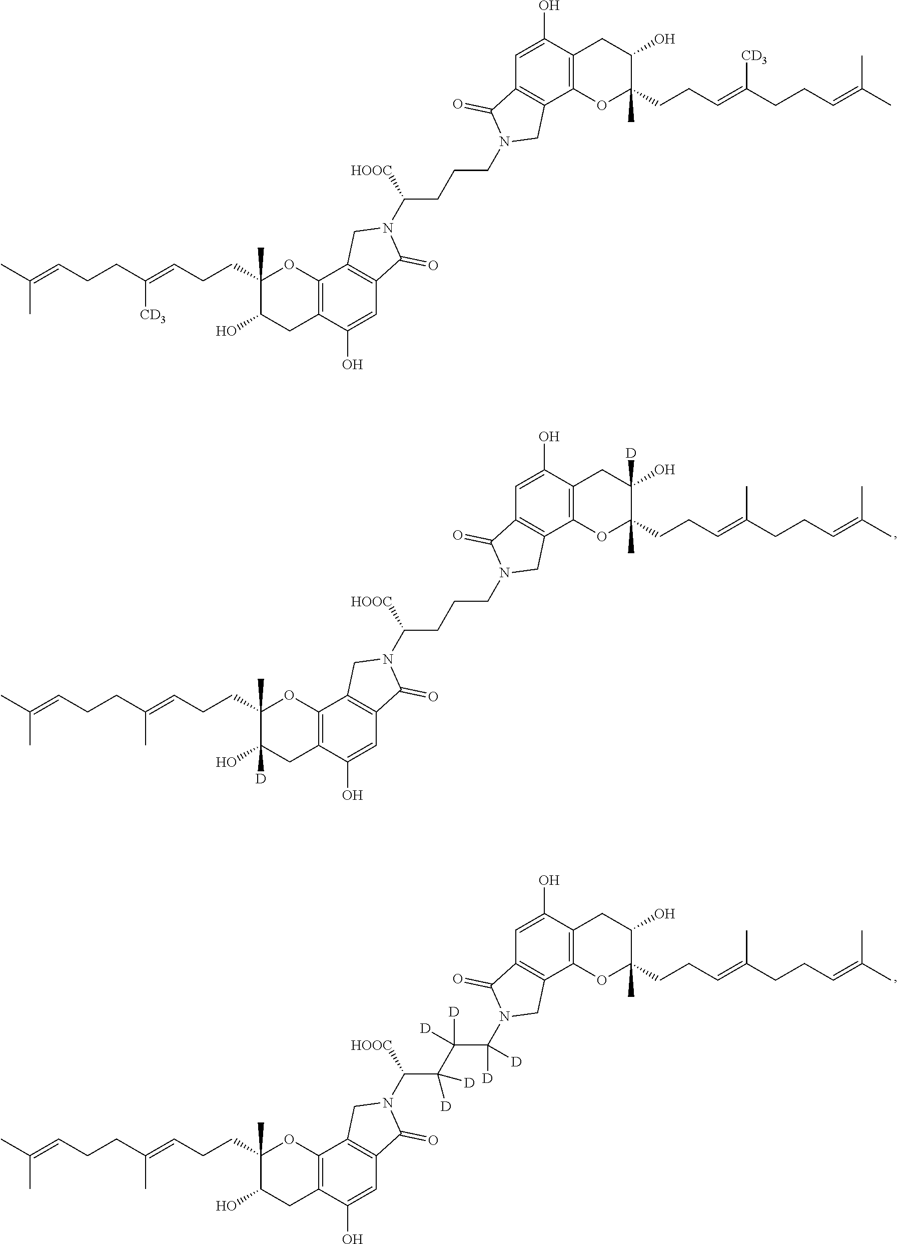

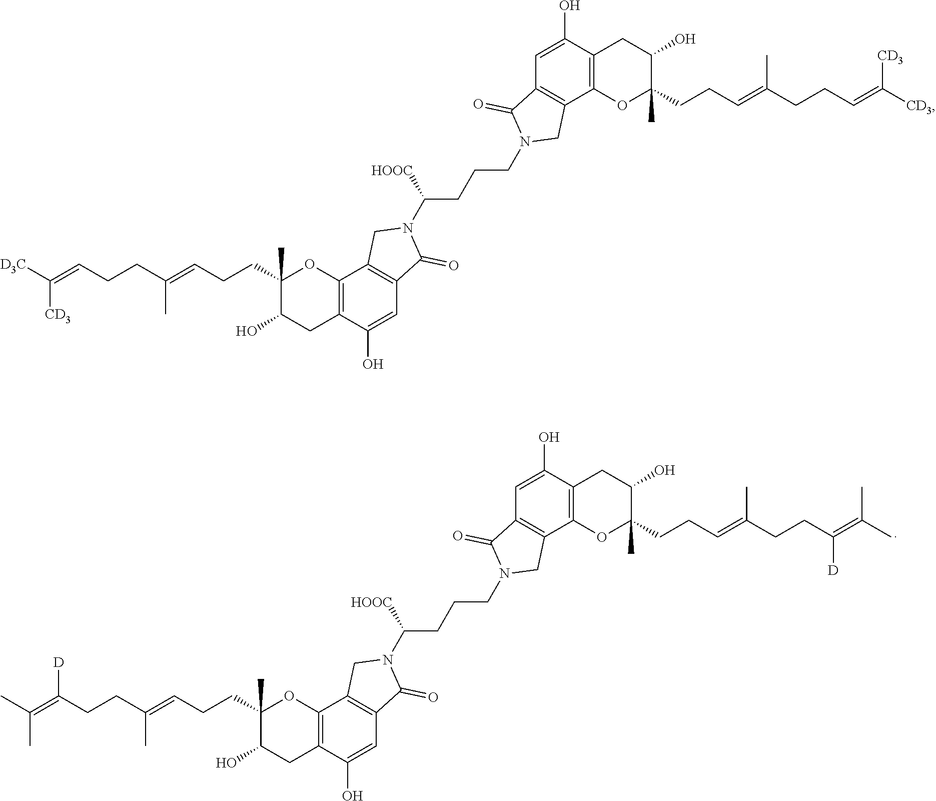 [SMILES: [2H]C([2H])([2H])C(=CCC/C(C)=C/CC[C@]1(C)Oc2c(c(O)cc3c2CN(CCC[C@@H](C(=O)O)N2Cc4c(cc(O)c5c4O[C@@](C)(CC/C=C(\C)CCC=C(C([2H])([2H])[2H])C([2H])([2H])[2H])[C@@H](O)C5)C2=O)C3=O)C[C@@H]1O)C([2H])([2H])[2H] ; [2H]C(CC/C(C)=C/CC[C@]1(C)Oc2c(c(O)cc3c2CN(CCC[C@@H](C(=O)O)N2Cc4c(cc(O)c5c4O[C@@](C)(CC/C=C(\C)CCC([2H])=C(C)C)[C@@H](O)C5)C2=O)C3=O)C[C@@H]1O)=C(C)C]

The disclosure also provides a pharmaceutical composition comprising a therapeutically effective amount of at least one of the compounds of formula I or the pharmaceutically acceptable salts thereof described above and a pharmaceutically acceptable excipient. In some embodiments, a unit dose of the pharmaceutical composition is 0.001 mg-1000 mg.

In certain embodiments, the pharmaceutical composition comprises 0.01-99.99% of the compound of formula (I) or the pharmaceutically acceptable salt thereof described above on the basis of the total weight of the composition. In certain embodiments, the pharmaceutical composition comprises 0.1-99.9% of the compound of formula I or the pharmaceutically acceptable salt thereof described above. In certain embodiments, the pharmaceutical composition comprises 0.5%-99.5% of the compound of formula I or the pharmaceutically acceptable salt thereof described above. In certain embodiments, the pharmaceutical composition comprises 1%-99% of the compound of formula I or the pharmaceutically acceptable salt thereof described above. In certain embodiments, the pharmaceutical composition comprises 2%-98% of the compound of formula I or the pharmaceutically acceptable salt thereof described above.

In certain embodiments, the pharmaceutical composition comprises 0.01%-99.99% of a pharmaceutically acceptable excipient based on the total weight of the composition. In certain embodiments, the pharmaceutical composition comprises 0.1%-99.9% of a pharmaceutically acceptable excipient. In certain embodiments, the pharmaceutical composition comprises 0.5%-99.5% of a pharmaceutically acceptable excipient. In certain embodiments, the pharmaceutical composition comprises 1%-99% of a pharmaceutically acceptable excipient. In certain embodiments, the pharmaceutical composition comprises 2%-98% of a pharmaceutically acceptable excipient.

The disclosure also provides a method for preventing and/or treating a cardiovascular or cerebrovascular disease, which comprises administering to a patient a therapeutically effective amount of the compound of formula I or the pharmaceutically acceptable salt thereof described above. In some embodiments, the disease is selected from a thromboembolic disease. In some embodiments, the disease is selected from the group consisting of myocardial infarction, angina pectoris, reocclusion and restenosis after angioplasty or aortic coronary artery shunt, disseminated intravascular coagulation, stroke, transient ischemic attack, peripheral arterial occlusive disease, pulmonary embolism and deep vein thrombosis.

The disclosure also provides use of the compound of formula I or the pharmaceutically acceptable salt thereof described above or the pharmaceutical composition described above is in the preparation of a medicament for preventing and/or treating a cardiovascular or cerebrovascular disease. In some embodiments, the disease is selected from a thromboembolic disease. In some embodiments, the disease is selected from the group consisting of myocardial infarction, angina pectoris, reocclusion and restenosis after angioplasty or aortic coronary artery shunt, disseminated intravascular coagulation, stroke, transient ischemic attack, peripheral arterial occlusive disease, pulmonary embolism and deep vein thrombosis.

The disclosure also provides the compound of formula I or the pharmaceutically acceptable salt thereof described above for preventing and/or treating a cardiovascular or cerebrovascular disease. In some embodiments, the disease is selected from a thromboembolic disease. In some embodiments, the disease is selected from the group consisting of myocardial infarction, angina pectoris, reocclusion and restenosis after angioplasty or aortic coronary artery shunt, disseminated intravascular coagulation, stroke, transient ischemic attack, peripheral arterial occlusive disease, pulmonary embolism and deep vein thrombosis.

In another aspect, the pharmaceutically acceptable salt of the compound in the disclosure is selected from the group consisting of an inorganic salt and an organic salt.

In another aspect, the compounds of the disclosure may exist in specific geometric or stereoisomeric forms. The disclosure contemplates all such compounds, including cis and trans isomers, (−)- and (+)-enantiomers, (R)- and (S)-enantiomers, diastereomers, (D)-isomer, (L)-isomer, and racemic mixtures and other mixtures thereof, such as enantiomerically or diastereomerically enriched mixtures, all of which are within the scope of the disclosure. Additional asymmetric carbon atoms may be present in substituents such as an alkyl group. All such isomers and mixtures thereof are included within the scope of the disclosure.

Optically active (R)- and (S)-enantiomers, and D- and L-isomers can be prepared by chiral synthesis, chiral reagents or other conventional techniques. If one enantiomer of a certain compound of the disclosure is desired, it may be prepared by asymmetric synthesis or derivatization with a chiral auxiliary, wherein the resulting mixture of diastereomers is separated and the auxiliary group is cleaved to provide the pure desired enantiomer. Alternatively, when the molecule contains a basic functional group (e.g., amino) or an acidic functional group (e.g., carboxyl), salts of diastereomers are formed with an appropriate optically active acid or base, followed by resolution of diastereomers by conventional methods known in the art, and the pure enantiomers are obtained by recovery.

In addition, separation of enantiomers and diastereomers is generally accomplished by chromatography using a chiral stationary phase, optionally in combination with chemical derivatization (e.g., carbamate formation from amines).

In the chemical structure of the compound of the disclosure, a bond “ [symbol] ” represents an is unspecified configuration—that is, if chiral isomers exist in the chemical structure, the bond “ [symbol] ” may be “ [symbol] ” or “ [symbol] ”, or contains both the configurations of “ [symbol] ” and “ [symbol] ”. In the chemical structure of the compound of the disclosure, a bond “ [symbol] ” is not specified with a configuration, that is, it may be in a Z configuration or an E configuration, or contains both configurations.

The compounds and intermediates of the disclosure may also exist in different tautomeric forms, and all such forms are included within the scope of the disclosure. The term “tautomer” or “tautomeric form” refers to structural isomers of different energies that can interconvert via a low energy barrier. For example, proton tautomers (also known as proton transfer tautomers) include interconversion via proton migration, such as keto-enol and imine-enamine, lactam-lactim isomerization. An example of a lactam-lactim equilibrium is present between A and B as shown below.

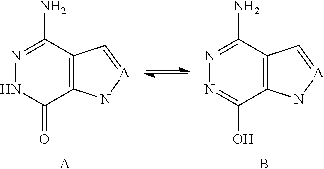

All compounds in the disclosure can be drawn as form A or form B. All tautomeric forms are within the scope of the disclosure. The nomenclature of the compounds does not exclude any tautomers.

The disclosure also includes isotopically-labeled compounds which are identical to those recited herein but have one or more atoms replaced by an atom having an atomic mass or mass number different from the atomic mass or mass number usually found in nature. Examples of isotopes that can be incorporated into the compound of the disclosure include isotopes of hydrogen, carbon, nitrogen, oxygen, phosphorus, sulfur, fluorine, iodine, and chlorine, such as ²H, ³H, ¹¹C, ¹³C, ¹⁴C , ¹³N , ¹⁵N , ¹⁵O, ¹⁷O, ¹⁸O, ³¹P, ³²P, ³⁵S, ¹⁸F, ¹²³I, ¹²⁵I and ³⁶Cl.

Unless otherwise stated, when a position is specifically designated as deuterium (D), that position shall be understood to be deuterium having an abundance that is at least 3000 times greater than the natural abundance of deuterium (which is 0.015%) (i.e., incorporating at least 45% deuterium). In certain embodiments, the abundance of each of the specified deuterium atoms in the compound of the disclosure is at least 3500 times (52.5% deuterium incorporation at each of the specified deuterium atoms), at least 4000 times (60% deuterium incorporation), at least 4500 times (67.5% deuterium incorporation), at least 5000 times (75% deuterium incorporation), at least 5500 times (82.5% deuterium incorporation), at least 6000 times (90% deuterium incorporation), at least 6333.3 times (95% deuterium incorporation), at least 6466.7 times (97% deuterium incorporation), at least 6600 times (99% deuterium incorporation) or at least 6633.3 times (99.5% deuterium incorporation) the natural abundance of deuterium. The disclosure also includes various deuterated forms of the compound of formula (I). Each available hydrogen atom connected to a carbon atom may be independently replaced by a deuterium atom. Those skilled in the art are able to synthesize the deuterated forms of the compound of formula (I) with reference to the relevant literature. Commercially available deuterated starting materials can be used in preparing the deuterated forms of the compound of formula (I), or they can be synthesized using conventional techniques with deuterated reagents including, but not limited to, deuterated borane, tri-deuterated borane in tetrahydrofuran, deuterated lithium aluminum hydride, deuterated iodoethane, deuterated iodomethane, and the like.

“Pharmaceutical composition” refers to a mixture containing one or more of the compounds or the physiologically or pharmaceutically acceptable salts or pro-drugs thereof described herein, and other chemical components, for example, physiologically or pharmaceutically acceptable carriers and excipients. The pharmaceutical composition is intended to promote the administration to an organism, so as to facilitate the absorption of the active ingredient, thereby exerting biological activity.

“Pharmaceutically acceptable excipient” or “acceptable excipient” includes, but is not limited to, any adjuvant, carrier, excipient, glidant, sweetener, diluent, preservative, dye/colorant, flavoring agent, surfactant, wetting agent, dispersant, suspending agent, stabilizer, isotonic agent, solvent or emulsifier that has been approved by the U.S. food and drug administration as acceptable for use in humans or livestock animals.

“Effective amount” or “therapeutically effective amount” described herein includes an amount sufficient to ameliorate or prevent a symptom or condition of a medical condition. An effective amount also refers to an amount sufficient to allow or facilitate diagnosis. The effective amount for a particular patient or veterinary subject may vary with factors such as the condition to be treated, the general health of the patient, the method and route and dosage of administration, and the severity of side effects. An effective amount may be the maximum dose or administration regimen to avoid significant side effects or toxic effects.

### DETAILED DESCRIPTION

The disclosure is further described below with reference to examples, which are not intended to limit the scope of the disclosure.

Experimental procedures without conditions specified in the examples of the disclosure were generally conducted according to conventional conditions, or according to conditions recommended by the manufacturers of the starting materials or commercial products. Reagents without origins specified are commercially available conventional reagents. The structures of the compounds were determined by nuclear magnetic resonance (NMR) spectroscopy and/or mass spectrometry (MS). NMR shifts (δ) are given in 10⁻⁶ (ppm). NMR analysis was performed on a Bruker AVANCE-400 nuclear magnetic resonance instrument, with deuterated dimethyl sulfoxide (DMSO-d₆), deuterated chloroform (CDCl₃) and deuterated methanol (CD₃OD) as solvents and tetramethylsilane (TMS) as an internal standard.

MS analysis was performed on an Agilent 1200/1290 DAD-6110/6120 Quadrupole MS liquid chromatography-mass spectrometry system (manufacturer: Agilent; MS model: 6110/6120 Quadrupole MS),

waters ACQuity UPLC-QD/SQD (manufacturer: waters, MS model: waters ACQuity Qda Detector/waters SQ Detector), and THERMO Ultimate 3000-Q Exactive (manufacturer: THERMO, MS model: THERMO Q Exactive).

High performance liquid chromatography (HPLC) analysis was performed using the following HPLC instruments: Agilent HPLC 1200DAD, Agilent HPLC 1200VWD and

Waters HPLC e2695-2489.

Chiral HPLC analysis was performed on an Agilent 1260 DAD high performance liquid chromatograph.

Preparative high performance liquid chromatography used Waters 2545-2767, Waters 2767-SQ Detecor2, Shimadzu LC-20AP and Gilson GX-281 preparative chromatographs. Preparative chiral chromatography used a Shimadzu LC-20AP preparative chromatograph. The CombiFlash preparative flash chromatograph used was CombiFlash Rf200 (TELEDYNE ISCO).

Yantai Huanghai HSGF254 or Qingdao GF254 silica gel plates, 0.15-0.2 mm layer thickness, were adopted for thin-layer chromatography (TLC) analysis and 0.4-0.5 mm layer thickness for TLC separation and purification.

Silica gel column chromatography generally used 200- to 300-mesh silica gel (Huanghai, Yantai) as the carrier.

Known starting materials described herein may be synthesized using or according to methods known in the art, or may be purchased from ABCR GmbH & Co. KG Acros Organics, Aldrich Chemical Company, Accela ChemBio Inc., Chembee Chemicals, and other companies.

In the examples, the reactions can all be performed in an argon atmosphere or a nitrogen atmosphere unless otherwise specified.

The argon atmosphere or nitrogen atmosphere means that the reaction flask is connected to a balloon containing about 1 L of argon or nitrogen.

The hydrogen atmosphere means that the reaction flask is connected to a balloon containing about 1 L of hydrogen.

Pressurized hydrogenation reactions were performed using a Parr 3916EKX hydrogenator and a Qinglan QL-500 hydrogenator, or an HC2-SS hydrogenator.

Hydrogenation reactions generally involve 3 cycles of vacuumization and hydrogen purging.

Microwave reactions were performed on a CEM Discover-S 908860 microwave reactor.

In the examples, a solution refers to an aqueous solution unless otherwise specified.

In the examples, reactions were conducted at room temperature, i.e., 20° C. to 30° C., unless otherwise specified.

The monitoring of the reaction progress in the examples was conducted by thin-layer chromatography (TLC). The developing solvent for reactions, the eluent system for column chromatography purification of compounds, the developing solvent system for thin-layer chromatography and the volume ratio of the solvents were adjusted according to the polarity of the compound, or by adding a small amount of basic or acidic reagents such as triethylamine and acetic acid.

### Example 1: Preparation of SMTP-7

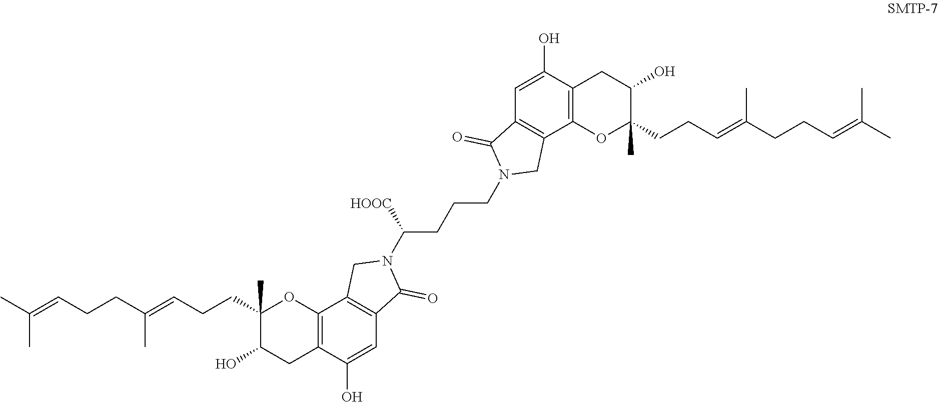 [SMILES: CC(C)=CCC/C(C)=C/CC[C@]1(C)Oc2c(c(O)cc3c2CN(CCC[C@@H](C(=O)O)N2Cc4c(cc(O)c5c4O[C@@](C)(CC/C=C(\C)CCC=C(C)C)[C@@H](O)C5)C2=O)C3=O)C[C@@H]1O]

Stachybotrys microspora IFO30018 was inoculated into a seed medium (4% glucose, 0.5% soybean meal, 0.3% dry broth, 0.3% yeast extract, 0.01% antifoaming agent, pH 5.8) and cultured for 4 days. The seed culture liquid was inoculated into a fermentation medium (5% sucrose, 0.1% yeast extract, 0.3% NaNO3, 0.1% K₂HPO₄, 0.05% MgSO₄·7H₂O, 0.05% KCl, 0.00025% CoCl₂·6H₂O, 0.0015% FeSO₄·7H₂O, 0.00065% CaCl₂·2H₂O, 0.01% antifoaming agent, pH 5.8). After 4 days of culture, L-ornithine was added; the culture was continued for 1 day, and fermentation was complete.

The fermentation broth was extracted with methanol. The extract was concentrated by rotary evaporation and extracted with ethyl acetate. The extract was dehydrated with anhydrous sodium sulfate, filtered, concentrated, dried and solidified.

The solid was dissolved in methanol, and the solution was subjected to pre-treatment and preparative purification using reversed-phase packing material. After steps such as ethyl acetate extraction, the target product was obtained.

### Example 2: Preparation of λ-Deutero-L-Ornithine (Compound 1e)

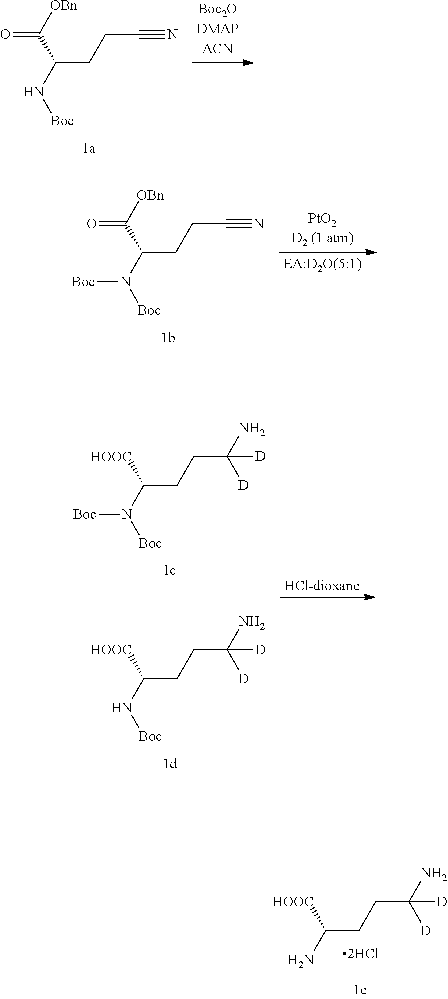 [SMILES: CC(C)(C)OC(=O)N[C@@H](CCC#N)C(=O)OCc1ccccc1 ; CC(C)(C)OC(=O)N(C(=O)OC(C)(C)C)[C@@H](CCC#N)C(=O)OCc1ccccc1 ; [2H]C([2H])(N)CC[C@@H](C(=O)O)N(C(=O)OC(C)(C)C)C(=O)OC(C)(C)C ; [2H]C([2H])(N)CC[C@H](NC(=O)OC(C)(C)C)C(=O)O ; [2H]C([2H])(N)CC[C@H](N)C(=O)O]

### Benzyl (S)-2-(bis(tert-butoxycarbonyl)amino)-4-cyanobutanoate (Compound 1b)

Compound 1a (prepared according to Synlett, 2016, vol. 27, 2, 309-312; 74.8 g, 234.9 mmol) was dissolved in acetonitrile (750 mL), Boc₂O (76.9 g, 352.4 mmol) was added, and DMAP (2.9 g, 23.5 mmol) was added. Then the reaction was conducted at 45° C. for 1-1.5 h. The reaction mixture was concentrated by rotary evaporation to remove the solvent. The crude product was purified by column chromatography to give the target compound 2b (95 g, 99.4% purity, 100% yield); MS (ESI) m/z 441.2 [M+Na]⁺.

### Compounds 1c and 1d

Compound 1b (4.18 g, 10 mmol) was dissolved in EA (100 mL) (super dry) and D₂O (20 mL), and PtO₂ (204 mg) was added. The system was purged with D₂, and the reaction was conducted in a D₂ atmosphere at 30° C. (external temperature) for 40 h. The reaction was substantially complete. The reaction mixture was separated, and the aqueous phase was washed with EA and then directly lyophilized to give a solid (1.87 g). The solid was dissolved in acetonitrile (5 mL) and ethyl acetate (20 mL). The solution was stirred at room temperature and filtered. The filter cake was washed with ethyl acetate and dried using an oil pump to give a mixture of compounds 1c and 1d (1.54 g, 46% yield).

### δ-Deutero-L-ornithine (Compound 1e)

The mixture of compounds 1c and 1d (195 mg, 0.58 mmol) was dissolved in a 6 M aqueous solution of hydrochloric acid, and the reaction was conducted at room temperature for 2 h. The reaction was substantially complete. The reaction mixture was concentrated by rotary evaporation to remove the solvent. The residue was dried to a constant weight using an oil pump to give a pale yellow solid product, compound 1e (120 mg, 100% yield, 97.25% purity).

HNMR (D₂O, 400M): 1.95-1.94 (m, 4H), 2.91-2.98 (m, 0.027H), 3.96 (t, J=6.4 Hz, 1H).

### Example 3: Preparation of δ-Deutero SMTP-7 (Compound 1)

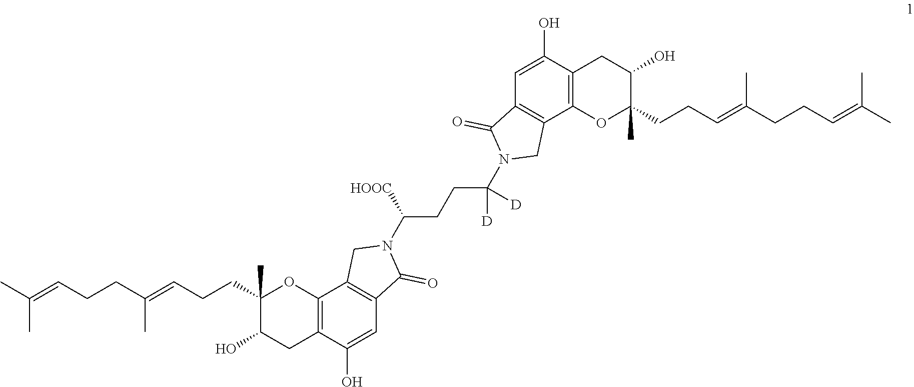 [SMILES: [2H]C([2H])(CC[C@@H](C(=O)O)N1Cc2c(cc(O)c3c2O[C@@](C)(CC/C=C(\C)CCC=C(C)C)[C@@H](O)C3)C1=O)N1Cc2c(cc(O)c3c2O[C@@](C)(CC/C=C(\C)CCC=C(C)C)[C@@H](O)C3)C1=O]

Compound 1 was prepared using δ-deutero L-ornithine (prepared according to Example 2) according to the method of Example 1.

Stachybotrys microspora IFO30018 was inoculated into a seed medium (4% glucose, 0.5% soybean meal, 0.3% dry broth, 0.3% yeast extract, 0.01% antifoaming agent, pH 5.8) and cultured for 4 days. The seed culture liquid was inoculated into a fermentation medium (5% sucrose, 0.1% yeast extract, 0.3% NaNO3, 0.1% K₂HPO_{4, 0.05}% MgSO₄·7H₂O, 0.05% KCl, 0.00025% CoCl₂·6H₂O, 0.0015% FeSO₄·7H₂O, 0.00065% CaCl₂·2H₂O, 0.01% antifoaming agent, pH 5.8). After 4 days of culture, δ-deutero L-ornithine was added; the culture was continued for 1 day, and fermentation was complete.

The fermentation broth was extracted with methanol. The extract was concentrated by rotary evaporation and extracted with ethyl acetate. The extract was dehydrated with anhydrous sodium sulfate, filtered, concentrated, dried and solidified.

The solid was dissolved in methanol, and the solution was subjected to pre-treatment and preparative purification using reversed-phase packing material. After steps such as ethyl acetate extraction, the target product was obtained.

¹H NMR (400 MHz, DMSO-d6) δ 13.07-12.66 (br., 1H), 9.79 (s, 1H), 9.73 (s, 1H), 6.66 (s, 1H), 6.62 (s, 1H), 5.28-5.09 (m, 3H), 5.07-4.95 (m, 2H), 4.72 (dd, J=9.9, 5.7 Hz, 1H), 4.26-4.05 (m, 4H), 3.73 (dd, J=13.0, 6.9 Hz, 2H), 2.82 (dt, J=17.0, 4.8 Hz, 2H), 2.48-2.38 (m, 2H), 2.17-2.05 (m, 4H), 2.04-1.95 (m, 4H), 1.95-1.81 (m, 6H), 1.66-1.46 (m, 23H), 1.18 (s, 3H), 1.15 (s, 3H).

### Test Example 1: Pharmacokinetic Study in Rats

### 1.1. Preparation of Test Samples

A proper amount of each of SMTP-7 and compound 1 was weighed out, and 2% (final volume) DMSO and 98% (final volume) normal saline were added. After they were well mixed by vortexing and ultrasonication, a 1 mg/mL clear solution was obtained for later use.

### 1.2. Animals

SD Rats, at the Age of 6-8 Weeks, Weighing about 180-220 g.

### 1.3. Regimen

| | | | | Concentration | |
|---|---|---|---|---|---|
| | Number | | | of test | |
| | of | Test | Dose | compound | Route of |
| Group | rats/sex | sample | (mg/kg) | (mg/mL) | administration |
| 1 | 3 rats/ | SMTP-7 | 5 | 1 | Intravenous |
| | male | | | | injection |
| 2 | 3 rats/ | Compound | 5 | 1 | Intravenous |
| | male | 1 | | | injection |

### 1.4. Sample Collection

About 0.20 mL of blood was collected for each sample via the jugular vein or other suitable is routes, and sodium heparin was used as the anticoagulant. The blood was placed on ice immediately after the collection. The collection was performed at a total of 10 time points: before administration, and 5 min, 0.25 h, 0.5 h, 1 h, 2 h, 4 h, 6 h, 10 h and 24 h after administration. The collected blood samples were placed in heparin anticoagulant blood collection tubes and centrifuged at 6800 g at 2-8° C. for 6 min to separate plasma. The plasma samples were stored in a freezer at −80° C. before analysis.

### 1.5. BiologicalAanalysis and Data Processing

The plasma concentration of each test compound was determined. The analysis of quality control samples was performed while the samples were analyzed, and more than 66.7% of the quality control samples were required to have an accuracy of 80-120%.

When plasma drug concentration-time curves were plotted, BLQ was recorded as 0. When the pharmacokinetic parameters were calculated, the concentration before administration was calculated as 0; BLQ before Cmax (including “No peak”) was calculated as 0; and BLQ that occurs after Cₘₐₓ (including “No peak”) was excluded from the calculation. The following pharmacokinetic parameters were calculated from the plasma concentration data of different time points using the non-compartmental analysis method of Phoenix WinNonlin 7.0 software: AUC₍₀₋ₜ₎, AUC_{(0-∞)}, T_{1/2}, MRT, Cₘₐₓ, Tₘₐₓ, etc.

### Experimental Results:

| Group | T_{1/2} (h) | Tₘₐₓ (h) | Cₘₐₓ (ng/ml) | AUC₍₀₋ₜ₎ (h*ng/ml) | MRT₍₀₋ₜ₎ |
|---|---|---|---|---|---|
| 1 | 6.689 ± 0.178 | 0.08 ± 0.00 | 26,311.98 ± 6,236.41 | 8,009.467 ± 1,832.168 | 0.465 ± 0.080 |
| 2 | 8.94 ± 0.61 | 0.08 ± 0.00 | 19,078.40 ± 4,312.75 | 11,287.80 ± 1,377.70 | 3.06 ± 0.25 |

Conclusion:
Compound 1 showed a longer half-life and a lower Cₘₐₓ than SMTP-7 after intravenous administration to SD rats.

### Example 4: Preparation of α-Deutero-L-ornithine

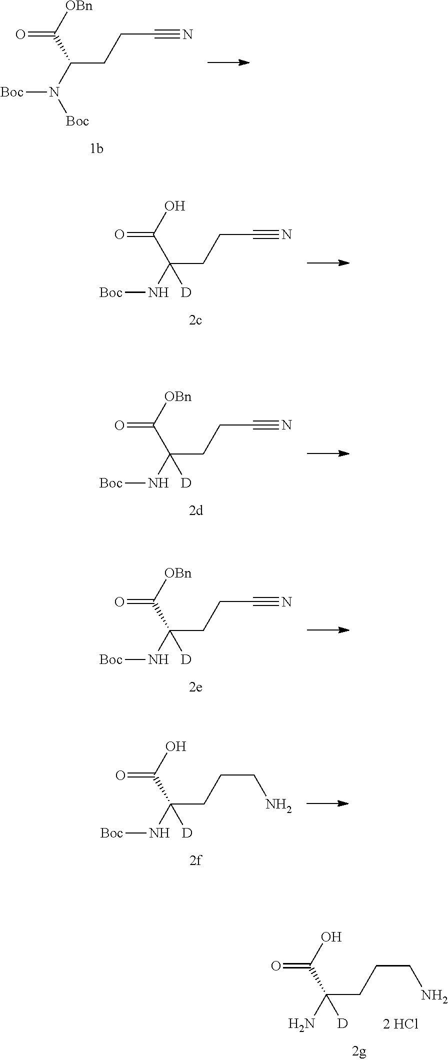 [SMILES: CC(C)(C)OC(=O)N(C(=O)OC(C)(C)C)[C@@H](CCC#N)C(=O)OCc1ccccc1 ; [2H]C(CCC#N)(NC(=O)OC(C)(C)C)C(=O)O ; [2H]C(CCC#N)(NC(=O)OC(C)(C)C)C(=O)OCc1ccccc1 ; [2H][C@](CCC#N)(NC(=O)OC(C)(C)C)C(=O)OCc1ccccc1 ; [2H][C@](CCCN)(NC(=O)OC(C)(C)C)C(=O)O ; [2H][C@@](N)(CCCN)C(=O)O]

### α-Deutero-2-((tert-butoxycarbonyl)amino)-4-cyanobutyric acid (Compound 2c)

In a nitrogen atmosphere, compound 1b (prepared according to Example 1; 25.0 g, 59.8 mmol) was added to 125 mL of MeOD and dissolved by stirring, and anhydrous potassium carbonate (41.3 g, 299.0 mmol) was added. The reaction was conducted at 20-30° C. for 16 h. The reaction was substantially complete. The system was directly concentrated to give a crude compound 2c. MS (ESI) m/z 252.1 [M+Na]⁺.

### Benzyl α-deutero-2-((tert-butoxycarbonyl)amino)-4-cyanobutanoate (Compound 2d)

To the crude compound 2c (25.0 g, 59.8 mmol) was added 150 mL of anhydrous acetonitrile followed by BnBr (15.3 g, 89.7 mmol). The reaction was conducted in a nitrogen atmosphere at 20-30° C. for 6 h. The reaction was substantially complete. The reaction mixture was filtered. The filter cake was washed with acetonitrile. The filtrate was concentrated, and the crude product was purified by column chromatography to give the target compound 2d (6.8 g), which was identified by chiral HPLC as a racemate (35.7% yield over two steps); MS (ESI) m/z 343.1 [M+Na]⁺.

### Benzyl α-deutero-(S)-2-((tert-butoxycarbonyl)amino)-4-cyanobutanoate (Compound 2e) The above-prepared racemic product, compound 2d (6.8 g), was chirally resolved to give compound 2e (3.6 g).

Instrument: MGII preparative SFC (SFC-14); chiral column: ChiralPak AY, 250×30 mm I.D., 5μm; mobile phase A: carbon dioxide; mobile phase B: methanol (0.1% ammonia water), gradient 15%; flow rate: 60 mL/min; back pressure: 100 bar; column temperature: 38° C.; detection wavelength: 220 nm; length of separation: about 6 min.

HNMR (CDC13, 400M): 1.44 (s, 9H), 1.58-1.65 (m,1H), 1.97-2.04 (m, 1H), 2.23-2.28 (m, 1H),, 2.38-2.46 (m, 1H), 5.17-5.20 (m, 2H), 7.36-7.38 (m, 5H).

### α-Deutero-(S)-5-amino-2-((tert-butoxycarbonyl)amino)pentanoic acid (Compound 2f)

Compound 2e (3.5 g, 10.9 mmol) was weighed out and dissolved in 70 mL of ethyl acetate, 700 mL of purified water was added, and PtO₂ (224 mg, 0.9 mmol) was added. The system was purged with hydrogen, and the reaction was conducted at 20-30° C. for 16 h. The reaction was substantially complete. The reaction mixture was filtered, and the filtrate was separated. The aqueous phase was collected, washed with water and lyophilized to give a crude product, the target compound 2f (about 1.6 g). The crude product was triturated with 20 mL of ethyl acetate and 2 mL of acetonitrile, and the triturate was filtered. The solid was dried under reduced pressure to give a purified product (about 1.5 g, 58.6% yield). MS-ESI: m/z 236.1 [M+H]+.

HNMR (D20, 400M):1.44 (s, 9H), 1.58-1.80 (m,4H), 2.93-2.98 (m, 2H).

### α-Deutero-L-ornithine (Compound 2g)

Compound 2f (1.5 g, 6.4 mmol) was weighed out and dissolved in about 10 mL of 6 M HCl by stirring. The reaction was conducted at 20-30° C. for 2 h. The reaction was substantially complete. The reaction mixture was directly lyophilized to give a crude compound 2g (1.2 g). The crude compound was triturated with 20 mL of acetonitrile, and the triturate was filtered to give a solid purified product (1.1 g, 83.9% yield). MS-ESI: m/z 134.1 [M+H]+. HNMR (D2O, 400M): 1.67-2.01 (m,4H), 2.98-3.01 (m, 2H).

### Example 5: Preparation of α-Deutero SMTP-7 (Compound 2)

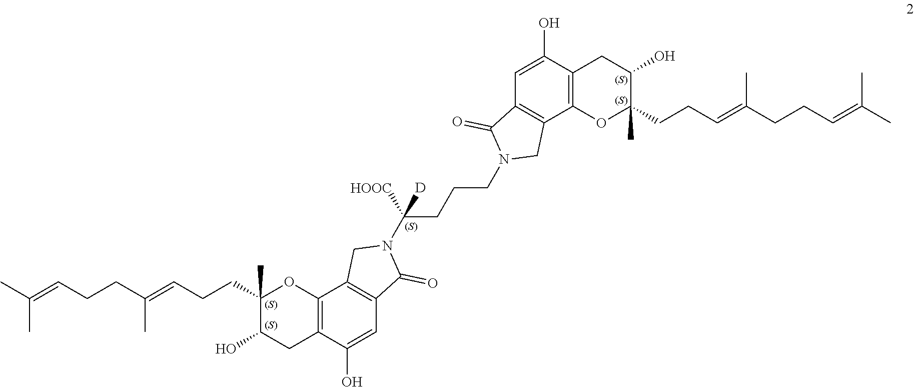

The target product, compound 2, was prepared using a-deutero L-ornithine (prepared according to Example 4) according to the method of Example 1.

### Example 6: Preparation of β-Deutero-L-ornithine (Compound 3e)

### β-Deutero-L-ornithine (Compound 3e)

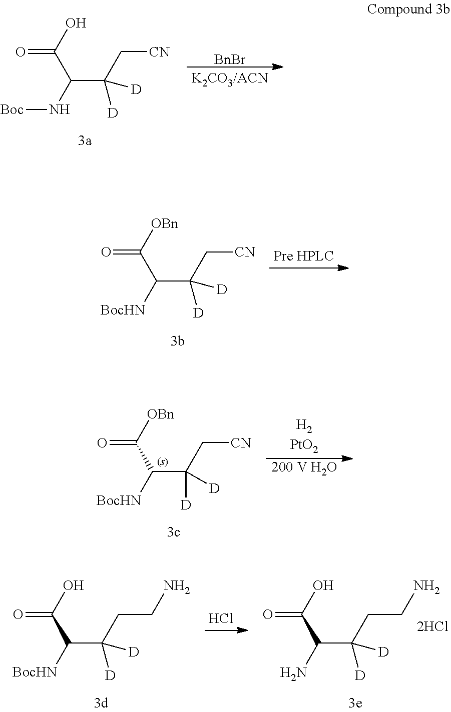

Compound 3a (3.8 g, 15.4 mmol, prepared according to Journal of the American Chemical Society, 2017, vol. 139, 39, 13830-13836) and anhydrous potassium carbonate (10.6 g, 77 mmol) were dissolved in 60 mL of anhydrous acetonitrile, and BnBr (5.3 g, 30.8 mmol) was added. The reaction was conducted at 10-20° C. for 16 h. The reaction mixture was filtered to remove insoluble matter. The organic phase was washed with EA. The filtrate was concentrated to give a crude product. The crude product was purified by column chromatography (PE:EA=10:1) to give the target compound 3b (4.8 g, 93.3% purity, 97% is yield).

MS-ESI: m/z 343.1 [M+Na]⁺

¹HNMR (CDCl₃, 400M):1.45 (s, 9H), 4.36-4.40 (m, 1H), 5.17-5.24 (s, 2H), 7.27-7.40 (m, 5H).

### Compound 3c

Compound 3b was a racemate. It was purified by preparative chiral chromatography to give a configuration monomer, the target compound 3c (2.2 g).

### Compound 3d

Compound 3c (2.2 g, 6.9 mmol) was dissolved in EA and water by stirring, and PtO2 (0.3 g, 1.3 mmol) was added. The system was purged with hydrogen three times, and the reaction was conducted at 10-20° C. for 16 h. The reaction mixture was filtered, and about 50 mL of EA was added to the aqueous phase. The mixture was separated, and the aqueous phase was collected, concentrated under reduced pressure and dried using an oil pump to give a crude product. The crude product was triturated at 10-20° C. with 20 mL of ethyl acetate and 2 mL of acetonitrile, and the triturate was filtered. The solid was washed with EA, collected and dried to give the target compound 3d (1.2 g).

MS-ESI: m/z 235.1 [M+H]⁺

### Compound 3e

Compound 3d (1.2 g, 5.1 mmol) was dissolved in 12 mL of a 6 M aqueous solution of hydrochloric acid. The reaction was conducted at 10-20° C. for 4 h. The reaction mixture was directly concentrated under reduced pressure using an oil pump to give the target compound 3e (1.05 g, 100% yield).

MS-ESI: m/z 135.1 [M+H]⁺

¹ HNMR (CDC₃, 400M):1.67-1.81 (m, 2H), 1.91-1.93 (m,0.1H), 2.95-2.32 (m, 2H), 3.98 (s, 1H).

### is Example 7: Preparation of β-Deutero SMTP-7 (Compound 3)

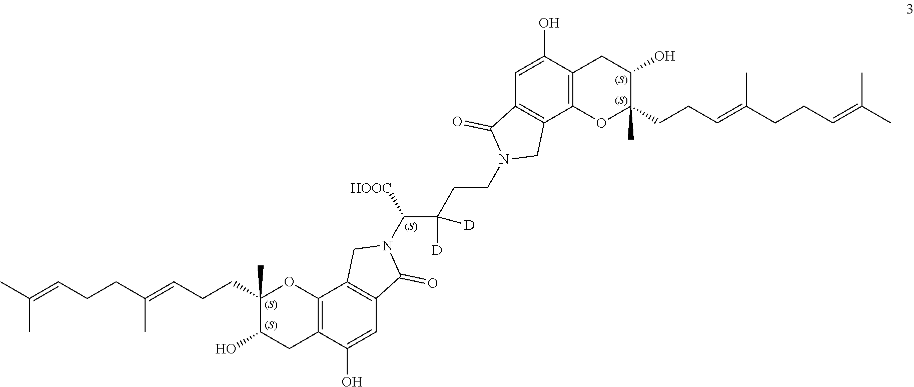

The target product, compound 3, was prepared using β-deutero L-ornithine (prepared according to Example 6) according to the method of Example 1.

### Example 8: Preparation of γ-Deutero-L-ornithine (Compound 4c)

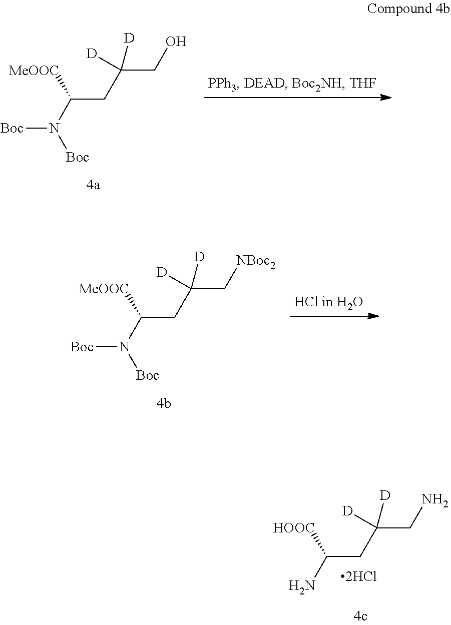

Compound 4a (2.2 g, 6.4 mmol, prepared according to Journal of the American Chemical

Society, 2018, vol. 140, 23, 7116-7126) was dissolved in tetrahydrofuran (50 mL), and Boc₂NH (2.1 g, 9.6 mmol) and triphenylphosphine (5.0 g, 19.1 mmol) were added. The mixture was cooled to 0° C., and DEAD (3.3 g, 19.1 mmol) was slowly added dropwise. After the addition, the reaction was warmed to room temperature and stirred overnight and was quenched with phosphate buffer. The reaction mixture was extracted with ethyl acetate. The organic phases were combined, washed with saturated brine, concentrated and purified by column chromatography (EA:PE=1:20) to give the target product, compound 4b (1.7 g, 50% yield).

¹H NMR (500 MHz, CDCl₃) δ4.87 (dd, J=9.5, 5.0 Hz, 1H), 3.70 (s, 3H), 3.59 (d, J=3.1 Hz, 2H), 2.09 (dd, J=14.2, 4.7 Hz, 1H), 1.88-1.81 (m, 1H), 1.49 (d, J=3.3 Hz, 36H).

### Compound 4c

Compound 4b (1.7 g, 3.2 mmol) and a 4 M aqueous solution of hydrochloric acid (30 mL) were added to a 100 mL sealed tube. The reaction was heated to 90° C. (external temperature) and was conducted for 16 h. The reaction mixture was cooled to room temperature, then concentrated and dried using an oil pump to give compound 4c (500 mg, is 92% yield).

MS-ESI: m/z 135.1 [M+H]⁺

¹ H NMR (500 MHz, D2O) δ3.99 (t, J=6.3 Hz, 1H), 3.03 (s, 2H), 1.97 (qd, J=14.5, 6.5 Hz, 2H).

### Example 9: Preparation of γ-Deutero SMTP-7 (Compound 4)

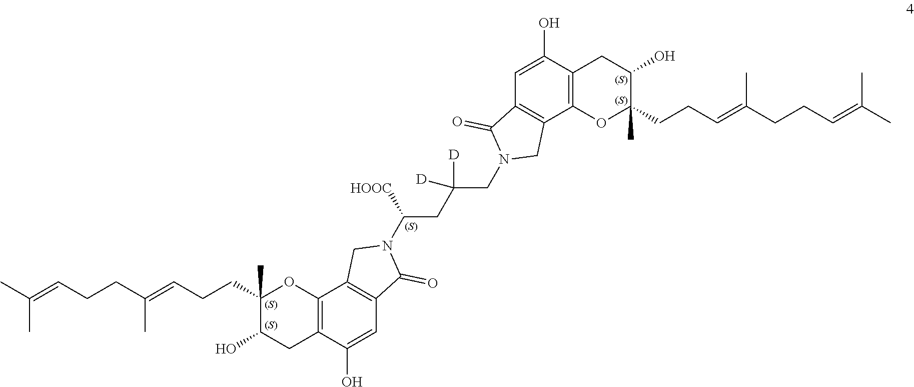

The target product, compound 4, was prepared using γ-deutero L-ornithine (prepared according to Example 8) according to the method of Example 1.

### Test Example 2: Pharmacokinetic Study in Rats

### 1.1. Preparation of Test Samples

A proper amount of each of SMTP-7, compound 2, compound 3 and compound 4 was weighed out, and normal saline was added (the pH was adjusted to about 9.2 with a 2 mM NaOH solution). After they were well mixed by vortexing and ultrasonication, a 1 mg/mL clear solution was obtained for later use.

### 1.2. Animals

SD Rats, at the Age of 6-8 Weeks, Weighing about 180-240 g.

1.3. Regimen

| | | Dose | Concentration of test | Route of |
|---|---|---|---|---|
| Group | Test sample | (mg/kg) | compound (mg/mL) | administration |
| 1 | SMTP-7 | 5 | 1 | Intravenous |
| | | | | injection |
| 2 | Compound 2 | 5 | 1 | Intravenous |
| | | | | injection |
| 3 | Compound 3 | 5 | 1 | Intravenous |
| | | | | injection |
| 4 | Compound 4 | 5 | 1 | Intravenous |
| | | | | injection |

The pharmacokinetic parameters such as AUC₍₀₋ₜ₎, AUC_{(0-∞)}, T_{1/2}, MRT, Cₘₐₓ and Tₘₐₓ were measured in rats according to the method of Test Example 1.

Experimental results: After intravenous administration to rats, SMTP-7, compound 2 and compound 3 did not greatly differ in T_{1/2} and AUC; compound 4 showed a lower T_{1/2} and a lower AUC than SMTP-7.

### Test Example 3: Thromboembolism in Rats

### 1.1. Preparation of a Cerebral Infarction Model

An embolic stroke model was prepared as the cerebral infarction model according to the method in the literature (J Cereb Blood Flow Metab, 1997, 17(2):123-135). 0.1 mL of rat blood was collected and immediately sucked into a PE50 catheter. The catheter was left at room temperature for 2 h and then stored at 4° C. for 22 h. The thrombus was pushed into 30 mL of normal saline and washed 3 times, for 5 min each time. A 5-mm length of embolus was cut out and sucked into a PE50 catheter with a special end for later use.

A rat was anesthetized with isoflurane and then fixed to an operating table in a supine position. A cut was made in the skin along the median line of the neck. The right common carotid artery was isolated, the internal carotid artery branch was isolated, and the pterygopalatine was clamped using an artery clip. A small cut was made in the common carotid artery, and the above embolus in the catheter was pushed into the cranium with 0.4 mL of normal saline. The catheter was carefully withdrawn. The common carotid artery was ligated, and the cut in the skin was sutured.

### 2.2. Grouping and Administration

Neurological function scoring was performed 1 h after the model was established. A score of ≥8 indicates that the model was successfully established. The rats were divided into a sham surgery group, a model control group, a test drug group (5, 10 and 20 mg/kg) and a control drug group (10 mg/kg). Each group included 10 rats. The sham surgery group and the model control group were administered normal saline. The test drug group was administered compound 1 (1 mg/mL, prepared in normal saline (the pH was adjusted to about 9.2 with a 2 mM NaOH solution)). The control drug group was administered SMTP-7 (1 mg/mL, prepared in normal saline (the pH was adjusted to about 9.2 with a 2 mM NaOH solution)). Intravenous administration was immediately performed 1 h after the model was established. 10% of the drug liquid was first injected, and the remaining 90% was infused over 30 min. The experiment ended 24 h after the administration.

### Neurological Function Scoring

The extent of behavioral disorders in the animals was observed and scored before administration and 24 h after treatment. The scoring criteria were as follows:

Criteria for scoring neurological function damage in MCAO rats

| Test | Performance | Score |
|---|---|---|
| 1. Motion testing | 1) Lift the tail of the rat | |
| | Flexion of the forelimbs | 1 |
| | Flexion of the hindlimbs | 1 |
| | Holds up its head within 30 s > 10. (with the vertical axis) | 1 |
| | 2) Allow the rat to walk on the floor (normal = 0; maximum = 3) | |
| | 0. Normally walks | 0 |
| | 1. Cannot walk straight | 1 |
| | 2. Circles to the hemiplegia side | 2 |
| | 3. Falls to the hemiplegia side | 3 |
| 2. Balance testing on a | Can balance in a stable posture | 0 |
| horizontal bar | Grasps the lateral sides of the horizontal bar | 1 |
| | Can hold the horizontal bar, but one limb falls off from the | 2 |
| | horizontal bar | |
| | Can hold the horizontal bar, but two limbs fall off from the | |
| | horizontal bar | 3 |
| | Or swings on the horizontal bar for over 60 s | |
| | Attempts to balance on the horizontal bar (>40 s), but falls off | 4 |
| | Attempts to balance on the horizontal bar (>20 s), but falls off | 5 |
| | Falls off, fails to balance, or does not want to struggle and hangs | 6 |
| | from the horizontal bar | |
| 3. Reflex testing | Auricular reflex (shakes its head when the entrance of the ear canal | 1 |
| | is touched) | |
| | Corneal reflex (blinks its eyes when the cornea is gently touched | 1 |
| | with cotton) | |
| | Startle reflex (produces a motion response when hearing the | 1 |
| | transient sound of tearing paper) | |
| | Seizures, myoclonus, dystonia | 1 |
| Total | | 16 |

### Cerebral Hemorrhage Assay

24 h after the scoring and blood collection, cardiac perfusion was performed and the brain was collected. The brain tissue was frozen in a −20° C. freezer and then sectioned from front to back; each section was 2 mm thick. A section scored 1 point if hemorrhage was present. The sum of the scores of the sections was the total hemorrhage score of each animal.

### Cerebral Infarction Range Assay

24 h after the scoring and blood collection, cardiac perfusion was performed and the brain was collected. The brain tissue was frozen in a −20° C. freezer and then sectioned from front to back; each section was 2 mm thick. The brain tissue sections were placed into a 2% solution of tetrazolium chloride (TTC) and incubated at 37° C. for 5 min. The infarcted tissues were white, and the non-infarcted tissues were red. The cerebral infarction area was measured using Image J software, and the percentage of the infarction area to the total brain area was calculated.

Percentage of cerebral infarction area %=cerebral infarction area/total brain area×100%

### Data Analysis Method

The metrology data are expressed as x̅±s, and pairwise comparisons were made using T-TEST; P<0.05 indicates a statistical difference.

### Experimental results:

TABLE 1
Behavioral scores and cerebral infarction area

| | | | Behavioral scores | | |
|---|---|---|---|---|---|
| | Dose | | Before | 24 h after | Infarction |
| Group | (mg/kg) | n | administration | administration | area % |
| Sham surgery | Normal saline | 10 | 0 ± 0 | 0 ± 0 | 0 ± 0 |
| Model group | Normal saline | 15 | 10.4 ± 0.5^{###} | 8.4 ± 2.1^{###} | 6.07 ± 4.98^{###} |
| | 5 | 10 | 10.5 ± 0.5 | 7.1 ± 1.8 | 2.47 ± 2.21* |
| Compound 1 | 10 | 10 | 10.2 ± 0.4 | 6.5 ± 2.5* | 1.38 ± 0.9** |
| | 20 | 10 | 10.2 ± 0.4 | 6.6 ± 2.2* | 1.7 ± 1.1* |
| SMTP-7 | 10 | 10 | 10.2 ± 0.4 | 6.3 ± 2.4* | 1.9 ± 2.35* |

Note:
^{#}means it is relative to the sham surgery group;
*means it is relative to the model group.

TABLE 2
Cerebral hemorrhage scores

| | | | Hemorrhage scores | |
|---|---|---|---|---|
| Group | Dose (mg/kg) | n | ≥1 (probability) | ≥3 (probability) |
| Sham surgery | Normal saline | 10 | 0 (0%) | 0 (0%) |
| Model group | Normal saline | 15 | 4 (27%) | 0 (0%) |
| | 5 | 10 | 2 (20%) | 0 (0%) |
| Compound 1 | 10 | 10 | 1 (10%) | 0 (0%) |
| | 20 | 10 | 1 (10%) | 0 (0%) |
| SMTP-7 | 10 | 10 | 2 (20%) | 1 (10%) |

In terms of behavior, when administered at the same dose (10 mg/kg), both compound 1 and SMTP-7 showed improvements compared to the model group and the improvements were comparable.

In terms of cerebral infarction area, both compound 1 and SMTP-7 showed significant improvements compared to the model group, and compound 1 was superior to SMTP-7 (a reduction of about 37%) when administered at the same dose of 10 mg/kg; the probability of serious infarction was relatively low.

In terms of cerebral hemorrhage, each dose group of compound 1 and SMTP-7 showed an improvement compared to the model group; the probability of cerebral hemorrhage after compound 1 (10%) was administered at the dose of 10 mg/kg was lower than that after SMTP-7 (20%) was administered at the dose of 10 mg/kg, and in addition, there was a cerebral hemorrhage score of ≥3 (10%) after SMTP-7 administration, which suggests that after SMTP-7 administration, there is a higher risk of hemorrhagic conversion in the brain, which is unfavorable for the clinical treatment of ischemic cerebral stroke.

Conclusion: Compound 1 has an improving effect on both the neurological function and the cerebral infarction area after cerebral infarction and involves a lower risk of hemorrhage.

1. A compound of formula I or a pharmaceutically acceptable salt thereof, 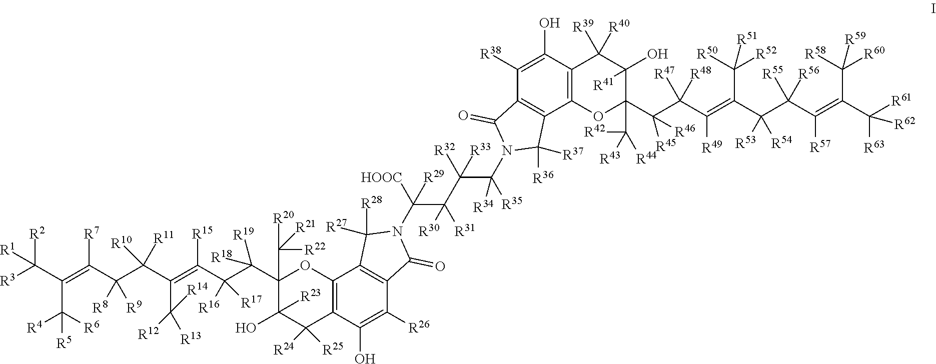
wherein F¹, R², R³, R⁴, R⁵, R⁶, R⁷, R⁸, R⁹, R¹⁰, R¹¹, R¹², R13 , R14 , R¹⁵, R¹⁶, R¹⁷, R¹⁸, R¹⁹,R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶² and R⁶³ are each independently hydrogen or deuterium, and at least one of R¹, R², R³, R⁴, R⁵, R⁶, R⁷, R⁸, R⁹, R¹⁰⁻, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶²and R⁶³ is deuterium.

2. (canceled)

3. (canceled)

4. (canceled)

5. The compound or the pharmaceutically acceptable salt thereof according to claim 1, wherein R²⁹ is selected from deuterium.

6. The compound or the pharmaceutically acceptable salt thereof according to claim 1, wherein R³⁰ is selected from deuterium; R³¹ is selected from deuterium.

7. The compound or the pharmaceutically acceptable salt thereof according to claim 1, wherein R³⁴ is selected from deuterium; R³⁵ is selected from deuterium.

8. (canceled)

9. (canceled)

10. (canceled)

11. (canceled)

12. The compound or the pharmaceutically acceptable salt thereof according to claim 1, wherein R³² is selected from deuterium; R³³ is selected from deuterium.

13. (canceled)

14. (canceled)

15. (canceled)

16. (canceled)

17. (canceled)

18. (canceled)

19. (canceled)

20. The compound or the pharmaceutically acceptable salt thereof according to claim 1, being

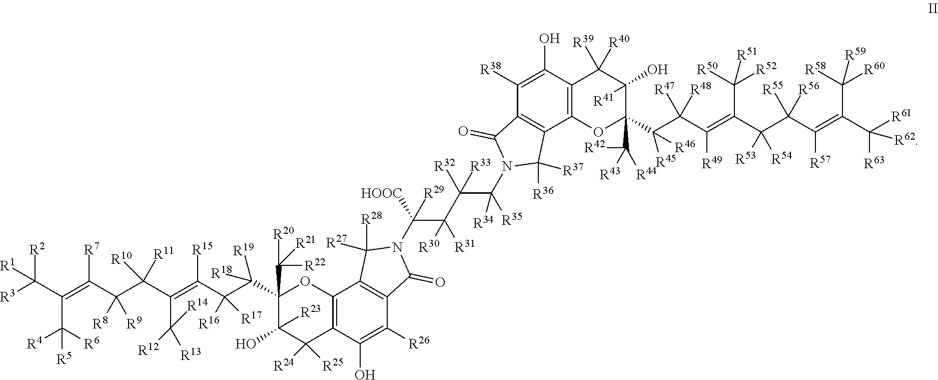

21. The compound or the pharmaceutically acceptable salt thereof according to claim 1, being selected from the group consisting of:

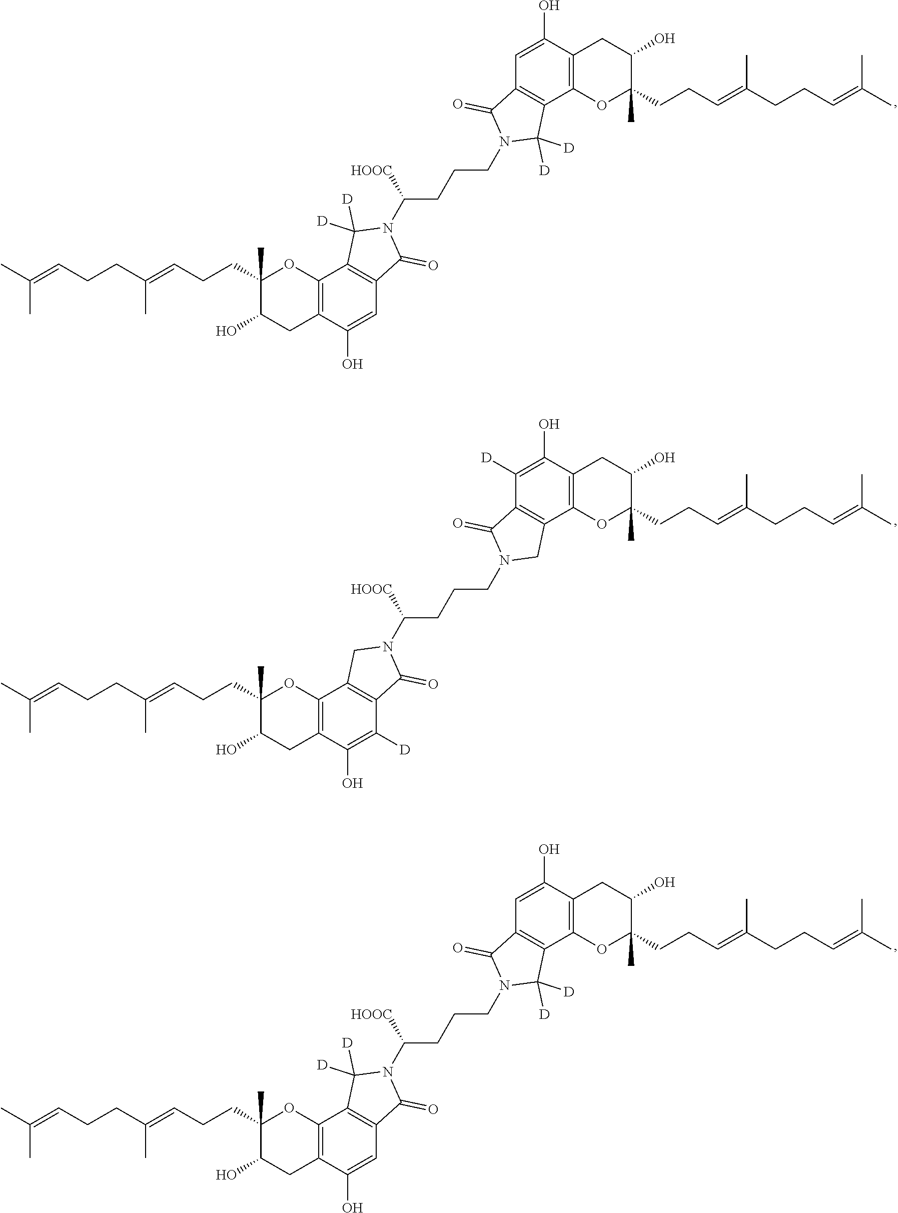

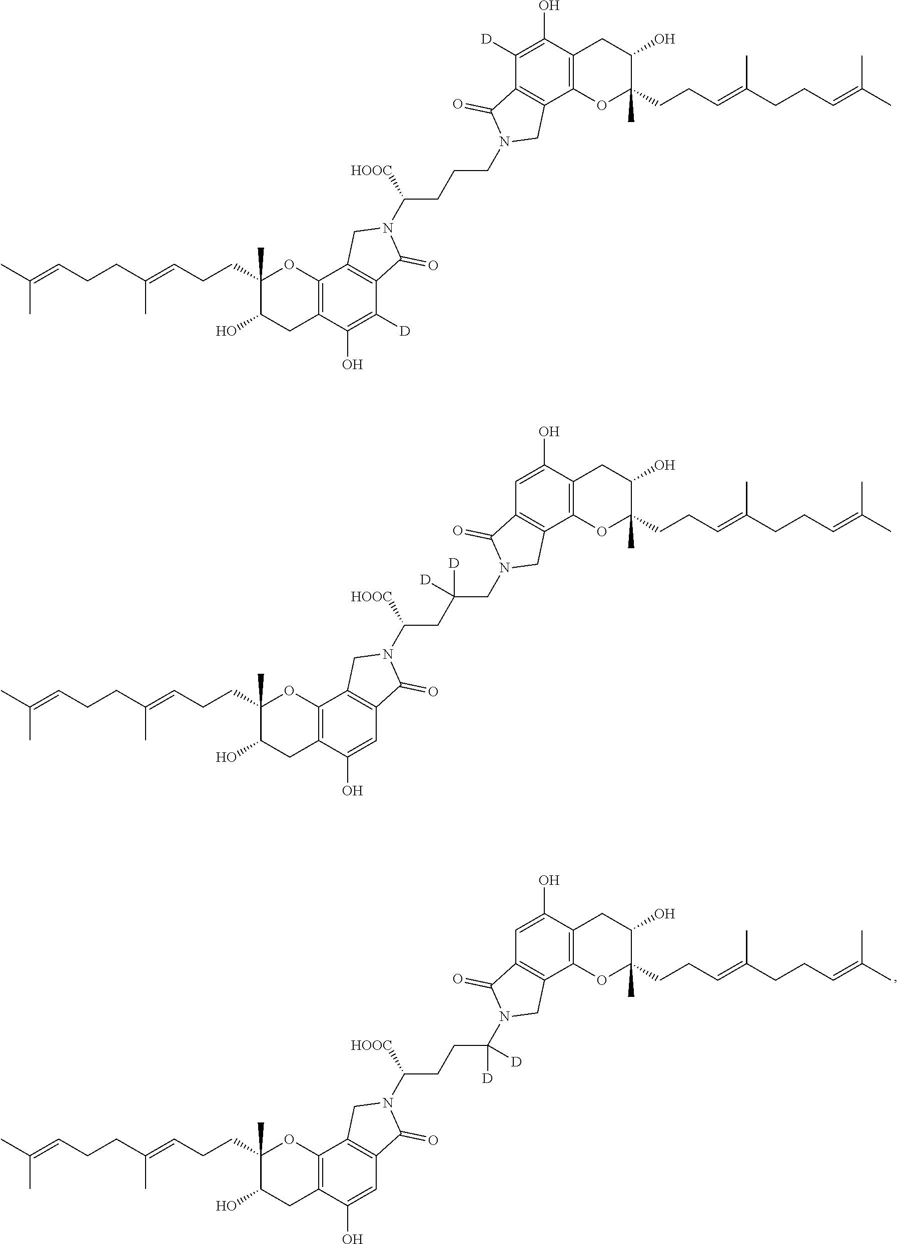

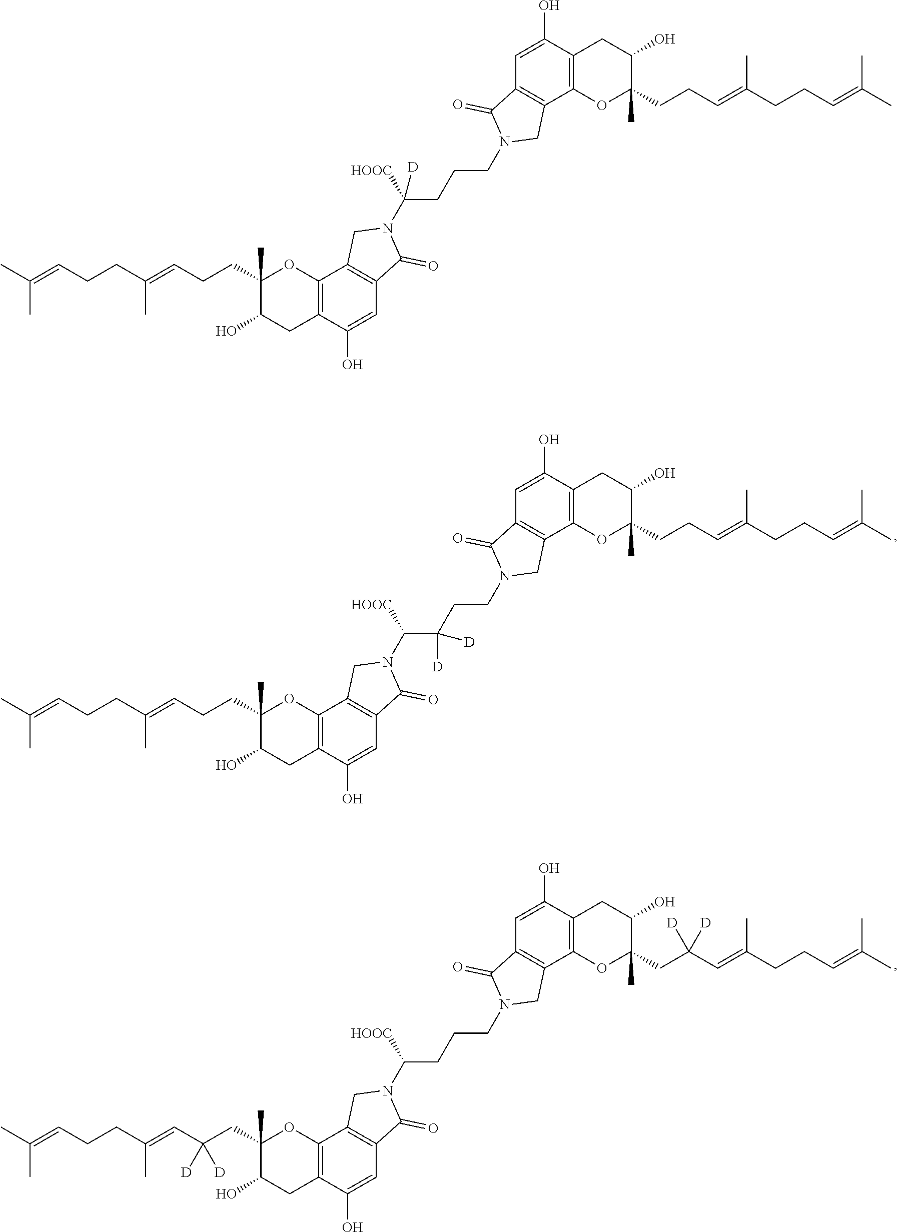

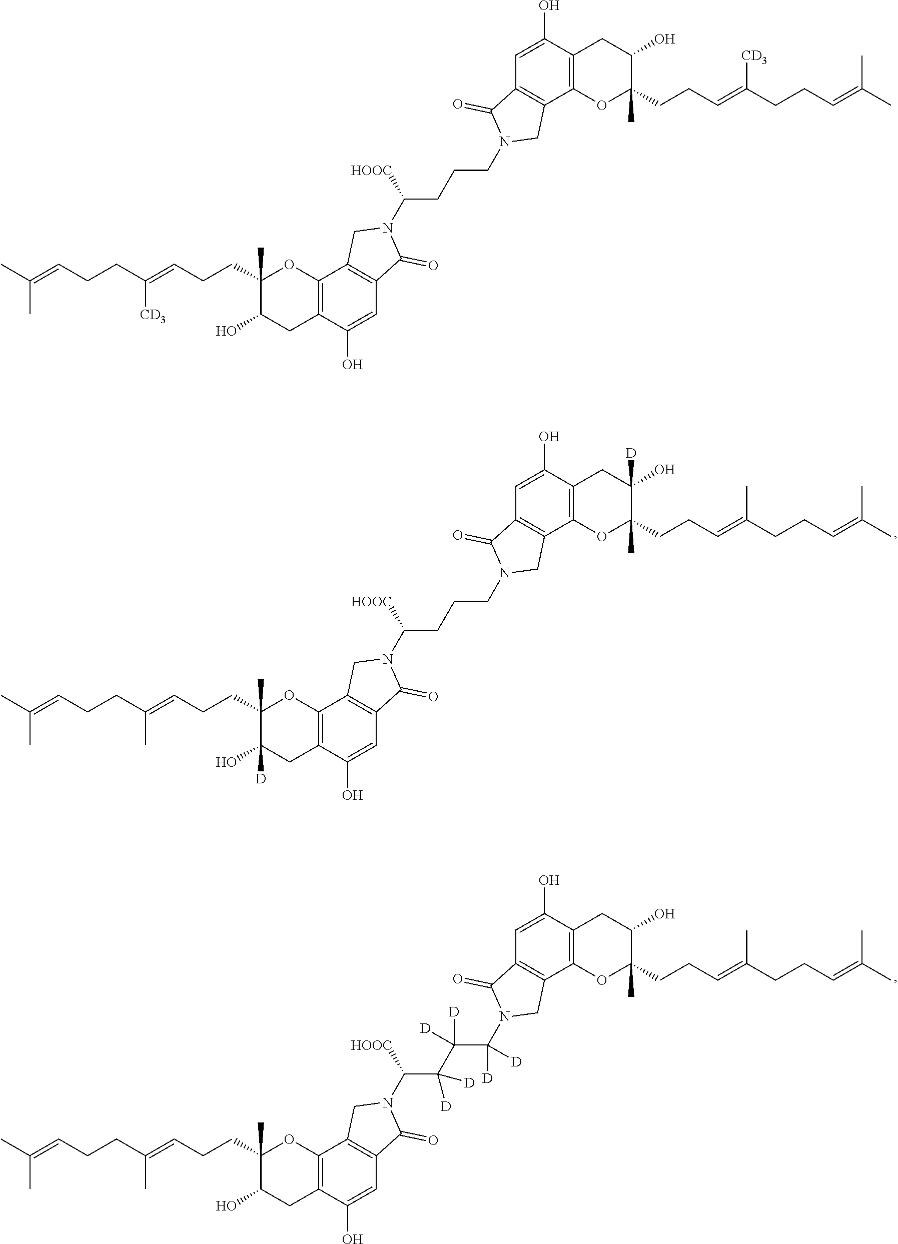

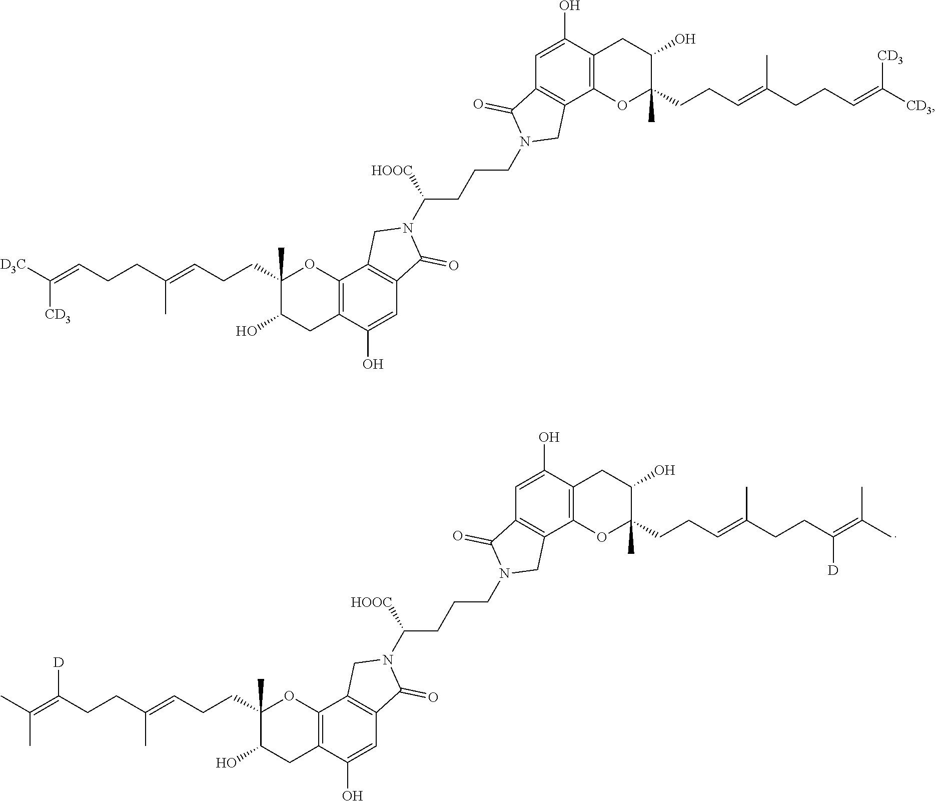 [SMILES: [2H]C([2H])([2H])C(=CCC/C(C)=C/CC[C@]1(C)Oc2c(c(O)cc3c2CN(CCC[C@@H](C(=O)O)N2Cc4c(cc(O)c5c4O[C@@](C)(CC/C=C(\C)CCC=C(C([2H])([2H])[2H])C([2H])([2H])[2H])[C@@H](O)C5)C2=O)C3=O)C[C@@H]1O)C([2H])([2H])[2H] ; [2H]C(CC/C(C)=C/CC[C@]1(C)Oc2c(c(O)cc3c2CN(CCC[C@@H](C(=O)O)N2Cc4c(cc(O)c5c4O[C@@](C)(CC/C=C(\C)CCC([2H])=C(C)C)[C@@H](O)C5)C2=O)C3=O)C[C@@H]1O)=C(C)C]

22. The compound or the salt thereof according to claim 1, wherein deuterium atoms have an abundance that is at least 4000 times.

23. A pharmaceutical composition comprising a therapeutically effective amount of at least one of the compound or the pharmaceutically acceptable salt thereof according to claim 1 and a pharmaceutically acceptable excipient.

24. A method for preventing and/or treating a cardiovascular or cerebrovascular disease, comprising administering to a patient a therapeutically effective amount of the compound or the pharmaceutically acceptable salt thereof according to claim 1-22.

25. (canceled)

26. The method according to claim 24, wherein the disease is a thromboembolic disease.

27. The method according to claim 24, wherein the disease is myocardial infarction, angina pectoris, reocclusion and restenosis after angioplasty or aortic coronary artery shunt, disseminated intravascular coagulation, stroke, transient ischemic attack, peripheral arterial occlusive disease, pulmonary embolism or deep vein thrombosis.
